(19) United States
(12) Patent Application Publication
(10) Pub. No.: US 2004/0071745 A1
(43) Pub. Date: Apr. 15, 2004

(54) Admixture of carotenoids and isoflavonoids for treating cutaneous symptoms of aging

(75) Inventors: Lionel Breton, Versailles (FR); Markus Baur, Ulm (DE); Christel Liviero, Paris (FR)

Correspondence Address: BURNS, DOANE, SWECKER & MATHIS, L.L.P., P.O. Box 1404, Alexandria, VA, 22313-1404, US

(73) Assignee: SOCIETE L'OREAL S.A., Paris (FR)

(21) Appl. No.: 10/424,128

(22) Filed: Apr. 28, 2003

(30) Foreign Application Priority Data
Oct. 26, 2000 (FR) FR 00/13755
Oct. 25, 2001 (WO) PCT/FR01/03319

(51) Int. Cl. A61K 31/7048; A61K 7/00; A61K 31/353; A61K 31/015

(52) U.S. Cl. 424/401; 514/27; 514/456; 514/763

(57) ABSTRACT

Intimate admixtures of at least one carotenoid and at least one isoflavonoid are well suited for treating the undesirable cutaneous signs of skin aging, in particular the determination of the skin and/or of the mucous membranes by inhibiting the activity and/or the expression of collagenases and by increasing the synthesis of collagen.

### CROSS-REFERENCE TO PRIORITY/PCT APPLICATIONS

This application claims priority under 35 U.S.C. § 119 of FR-00/13755, filed Oct. 26, 2000, and is a continuation of PCT/FR01/03319, filed Oct. 25, 2001 and designating the United States (published in the French language on May 2, 2002 as WO 02/034233 A3; the title and abstract were also published in English), both hereby expressly incorporated by reference.

### CROSS-REFERENCE TO COMPANION APPLICATIONS

Copending application Ser. No. ______ [Attorney Docket No. 016800-604] and Ser. No. ______ [Attorney Docket No. 016800-605], both filed concurrently herewith and both also hereby expressly incorporated by reference.

### BACKGROUND OF THE INVENTION

1. Technical Field of the Invention

The invention relates to the use, in a composition or for the preparation of a composition, of the combination of at least one carotenoid and at least one isoflavonoid, the combination or the composition being intended for treating the cutaneous signs of aging, and in particular the deterioration of the skin and/or of the mucous membranes by inhibiting the activity and/or the expression of collagenases and by increasing the synthesis of collagen.

The subject of the invention is also a method for the cosmetic treatment of the skin and/or of the mucous membranes.

2. Description of Related/Prior Art

In mammals in general, particularly in humans, the skin consists of two compartments, namely a compartment which is in contact with the outside, the epidermis, and a deep compartment which serves as a support for the epidermis, the dermis.

The epidermis is mainly composed of three types of cell which are the keratinocytes, highly predominant, the melanocytes and the Langerhans' cells. Each of these cell types contributes, through its specific functions, to the essential role which the skin plays in the body.

The dermis provides the epidermis with a solid support. It is also its feeder component. It consists mainly of fibroblasts and an extracellular matrix itself mainly composed of collagen, elastin and a substance, called ground substance, components synthesized by the fibroblast. Also present therein are leukocytes, mastocytes or tissue macrophages. It is also traversed by blood vessels and nerve fibers. In a normal skin, that is to say with no pathology or wound healing, the fibroblast is in the quiescent state, that is to say is nonproliferative, not very active from a metabolic point of view and not mobile. The collagen fibers are predominantly responsible for the solidity of the dermis. These fibers consist of fibrils firmly attached to each other, thus forming more than ten types of different structures. The solidity of the dermis is predominantly due to the entanglement of the collagen fibers packed against each other in every direction. The collagen fibers also participate in the elasticity and especially in the tonicity of the skin and/or of the mucous membranes.

The collagen fibers are constantly renewed, but this renewal decreases with age, which causes a reduction in the thickness of the dermis. This reduction in the thickness of the dermis is also due to pathological causes such as, for example, the hyper-secretion of corticoid hormones, certain pathologies or vitamin deficiencies. It is also accepted that extrinsic factors such as ultraviolet rays, tobacco or certain treatments (Glucocorticoids, vitamin D and derivatives, for example) also have an effect on the skin and on its collagen level.

However, various factors cause the degradation of collagen with all the consequences which may be envisaged on the structure and/or the firmness of the skin and/or the mucous membranes.

Although they are very resistant, collagen fibers are susceptible to certain enzymes called collagenases. Degradation of the collagen fibers causes the appearance of a soft and wrinkled skin which it has always been sought to combat, particularly in human beings, who prefer the appearance of a smooth and tight skin.

Collagenases form part of a family of enzymes called metalloproteinases (MMPs) which are themselves members of a family of proteolytic enzymes (endoproteases) which possess a zinc atom coordinated to 3 cysteine residues and a methionine in their active site and which degrade the macromolecular components of the extracellular matrix and of the basal laminae at neutral pH (collagen, elastin, and the like). Being very widely distributed in the living world, these enzymes are present, but weakly expressed, in normal physiological situations such as the growth of organs and the renewal of tissues.

Their overexpression and their activation are however linked to numerous processes, sometimes pathological, which involve the destruction and the remodeling of the matrix. That causes either an uncontrolled resorption of the extracellular matrix, or conversely the establishment of a state of fibrosis.

The family of metalloproteinases consists of several well defined groups based on their resemblance in terms of structure and substrate specificity (see Woessner J. F., Faseb Journal, vol. 5, 1991, 2145). Among these groups, there may be mentioned the collagenases intended to degrade fibrillary collagens (MMP-1 or interstitial collagenase, MMP-8 or neutrophil collagenase, MMP-13 or collagenase 3), gelatinases which degrade type IV collagen or any form of denatured collagen (MMP-2 or gelatinase A (72 kDa), MMP-9 or gelatinase B (92 kDa)), stromelysins (MMP-3) whose broad activity spectrum affects the proteins of the extracellular matrix such as glycoproteins (fibronectin, laminin), proteoglycans, and the like, or alternatively membrane metalloproteinases.

Prolonged exposure to ultraviolet radiation, particularly type A and/or B ultraviolet radiation, has the effect of stimulating the expression of collagenases, particularly of MMP-1. That is one of the components of photoinduced skin aging.

Moreover, at menopause, the principal modifications relating to the dermis are a decrease in the collagen level and in the dermal thickness. This causes, in menopausal women, a reduction in the thickness of the skin and/or of the mucous membranes. Women then experience a sensation of “dry skin” or of tight skin and an accentuation of the surface wrinkles and fine lines is observed. The skin exhibits a rough appearance upon palpation. Finally, the skin exhibits a reduced suppleness.

It is thus possible to understand, on reading the preceding text, the importance of collagen in the structure of tissues, particularly of the skin and/or the mucous membranes, and the importance there is to combat its degradation and thereby control the cutaneous signs of aging, whether it is chronological or photoinduced, and its consequences, such as for example the reduction in the thickness of the dermis and/or the degradation of collagen fibers, which causes the appearance of a soft and wrinkled skin.

The expression cutaneous signs of aging is understood to mean any modifications in the external appearance of the skin due to aging, whether it is chronobiological and/or photoinduced, such as, for example, wrinkles and fine lines, withered skin, soft skin, skin with reduced thickness, dull skin and skin with no brightness, lack of elasticity and/or of tone of the skin, but also all the internal modifications of the skin which do not systematically result in a modified external appearance, such as, for example, all the internal damage to the skin, particularly to the collagen fibers, following exposure to ultraviolet radiation, which may have the consequence of reducing the thickness of the dermis.

### SUMMARY OF THE INVENTION

The present invention thus provides a product which makes it possible to treat, in mammals in general, particularly in humans, preventively and/or curatively, the cutaneous signs of aging, whether it is chronobiological or photoinduced, particularly the reduction in the thickness of the dermis and/or the degradation of collagen fibers, by a stimulatory effect on the synthesis of collagen and/or an inhibitory effect on collagenases and if possible no notable side effects.

Surprisingly, it has now been determined that the combination (intimate admixture) of at least one carotenoid and at least one isoflavonoid makes it possible to preventively and/or curatively treat the cutaneous signs of aging, whether it is chronobiological or photoinduced, particularly the reduction in the thickness of the dermis and/or the degradation of collagen fibers, by a stimulatory effect on the synthesis of collagen and/or an inhibitory effect on collagenases.

One of the remarkable properties of the combination of the invention is that it has effects in higher proportions than those reasonably expected from the mere addition of the effects of each of these components taken separately.

One advantage of this property is to allow a use, in the composition of the invention, of a quantity of each of the products which is less than is generally acceptable to use.

To date, the stimulatory activity on the synthesis of collagen and the inhibitory activity on the expression and/or the activity of collagenases of the combination of at least one carotenoid and at least one isoflavonoid has never been described.

Accordingly, the first aspect of the invention is the use, in a composition or for the preparation of a composition, of the combination of at least one carotenoid and at least one isoflavonoid, the combination or the composition being intended for treating (regime or regimen) the cutaneous signs of aging.

The second aspect of the invention is the use, in a composition or for the preparation of a composition, of the combination of at least one carotenoid and at least one isoflavonoid, the combination or the composition being intended for combating the degradation of collagen.

The third aspect of the invention is the use, in a composition or for the preparation of a composition, of the combination of at least one carotenoid and at least one isoflavonoid, the combination or the composition being intended for inhibiting the expression of the proteases of the extracellular matrix, particularly of metalloproteinases and still more particularly of metalloproteinase type 1.

The fourth aspect of the invention is the use of the combination in a composition or for the preparation of a composition, of the combination of at least one carotenoid and at least one isoflavonoid, the combination or the composition being intended for treating skin conditions linked to aging, in particular to skin conditions of the menopause.

The fifth aspect of the invention is the use, in a composition or for the preparation of a composition, of the combination of at least one carotenoid and at least one isoflavonoid, the combination or the composition being intended for combating wrinkles and fine lines.

The sixth aspect of the invention is the use, in a composition or for the preparation of a composition, of the combination of at least one carotenoid and at least one isoflavonoid, the combination or the composition being intended for combating withered skin.

The seventh aspect of the invention is the use, in a composition or for the preparation of a composition, of the combination of at least one carotenoid and at least one isoflavonoid, the combination or the composition being intended for combating soft skin.

The eighth aspect of the invention is the use, in a composition or for the preparation of a composition, of the combination of at least one carotenoid and at least one isoflavonoid, the combination or the composition being intended for combating skin with reduced thickness.

The ninth aspect of the invention is the use, in a composition or for the preparation of a composition, of the combination of at least one carotenoid and at least one isoflavonoid, the combination or the composition being intended for combating dull skin or skin with no brightness.

The tenth aspect of the invention is the use, in a composition or for the preparation of a composition, of the combination of at least one carotenoid and at least one isoflavonoid, the combination or the composition being intended for combating the lack of elasticity and/or of tone of the skin.

### DETAILED DESCRIPTION OF BEST MODE AND SPECIFIC/PREFERRED EMBODIMENTS OF THE INVENTION

Whatever the envisaged use of the invention, it may be preventively and/or curatively for the condition which it aims to treat.

It is apparent that the invention is intended for mammals in general and particularly human beings.

The expression carotenoid is understood to mean, according to the invention, both a carotenoid with provitamin A activity and a carotenoid without provitamin A activity.

Of course, according to the invention, the carotenoid may be a mixture of carotenoids with provitamin A activity and of carotenoids without provitamin A activity. This mixture may be in any proportion.

According to the invention, the carotenoid with provitamin A activity may be a mixture of carotenoids with provitamin A activity. This mixture may be in any proportion. Among the carotenoids with provitamin A activity, there may be mentioned, by way of example, β-carotene or α-carotene.

Preferably, β-carotene is used according to the invention.

According to the invention, the carotenoid without provitamin A activity may be a mixture of carotenoids without provitamin A activity. This mixture may be in any proportion. Among the carotenoids without provitamin A activity, there may be mentioned, by way of example, zeaxanthin, cryptoxanthin, lutein or lycopene or a mixture thereof.

Preferably, lycopene is used according to the invention.

Preferably, according to the invention, a carotenoid without provitamin A activity, and still more preferably lycopene, is used.

Lycopene is a natural pigment which is found in ripe fruits, particularly in tomato. It belongs to the family of carotenoids and its structure is close to that of β-carotene.

The role of lycopene in the maturation of fruits is known in the prior art.

Lycopene is used in compositions with tanning activity for its role on the synthesis of melanin (WO 97/47278), in compositions intended for the treatment of hair and/or acne, for its activity on 5α-reductases (JP-2940964) or as an anti-free radical agent (JP-A-8-283136).

Lycopene may be in the cis or trans chemical form.

The carotenoid used according to the invention may be of natural or synthetic origin. The expression natural origin is understood to mean the carotenoid, in the pure state or in solution of whatever its concentration in said solution, obtained from a natural component.

According to a preferred embodiment of the invention, a lycopene-rich extract, such as for example a tomato extract, is used.

The expression synthetic origin is understood to mean the carotenoid, in the pure state or in solution whatever its concentration in said solution, obtained by chemical synthesis.

When the carotenoid is of natural origin, it may be obtained from a plant material derived from the whole plant cultivated in vivo or derived from cultivation in vitro.

The expression cultivation in vivo is understood to mean any culture of the conventional type, that is to say in the soil in the open air or in a greenhouse, or alternatively with no soil.

The expression cultivation in vitro is understood to mean all the techniques known to persons skilled in the art which make it possible to artificially obtain a plant or a portion of a plant. The selection pressure imposed by the physicochemical conditions during the growth of plant cells in vitro make it possible to obtain a standard plant material which is available throughout the year, unlike the plants cultivated in vivo.

Preferably, according to the invention, a plant derived from cultivation in vivo is used.

Any method of extraction known to persons skilled in the art may be used to prepare the carotenoid used according to the invention.

The carotenoid may be in alcoholic, in particular ethanolic, solution.

The carotenoid may also be in a lipid (oil) or lipoalcoholic solution.

By way of example, according to the invention, there is used a lycopene-rich tomato extract, prepared by the company Metaphar, marketed under the name LycOMato® consisting of an oleoresin extract (fatty phase) containing 6% of pure lycopene.

It is also possible to use according to the invention any preparation containing lycopene having the objective of improving the bioavailability of the latter and/or all the novel methods for the manufacture and/or formulation and/or encapsulation of lycopene.

The quantity of carotenoid which can be used according to the invention of course depends on the desired effect and may therefore vary to a large degree.

To give an order of magnitude, in the composition according to the invention, the carotenoid in the pure state is in a quantity representing from 10-12% to 20% of the total weight of the composition and preferably in a quantity representing from 10⁻¹⁰% to 10% of the total weight of the composition.

Of course persons skilled in the art, if they use the carotenoid in the form of a solution, a plant extract for example, know how to adjust the quantity of solution which they use in its composition so that the final quantity of carotenoid in the composition is consistent with the above-defined quantities which can be used.

The isoflavonoids constitute a subclass of flavonoids, consisting of a 3-phenylchroman backbone which may contain varied substituents and different oxidation levels. Unlike the flavonoids, they are only present in a very limited number of plants.

The term isoflavonoid groups together several classes of compounds, among which there may be mentioned isoflavones, isoflavanones, retinoids, pterocarpans, isoflavans, isoflavan-3-enes, 3-aryl-coumarins, 3-aryl-4-hydroxycoumarins, coumestans, coumaronochromones, α-methyldeoxybenzoins or 2-aryl-benzofurans.

In this regard, reference may be advantageously made, for a complete review, to isoflavonoids, methods for their analysis and their sources, in chapter 5 “Isoflavonoids” written by P. M. Dewick, in The Flavonoids, Harbone editor, pp. 125-157 (1988).

The isoflavonoids which are suitable for use in the present invention may be of natural or synthetic origin. The expression “natural origin” is understood to mean the isoflavonoid in the pure state or in solution at different concentrations, obtained by different methods of extraction from a component, generally a plant of natural origin. The expression “synthetic origin” is understood to mean isoflavonoid in the pure state or in solution at different concentrations, obtained by chemical synthesis.

It is preferable to use isoflavonoids of natural origin. Among these, there may be mentioned: daidzein, formononetin, cuneatin, genistein, isoprunetin and prunetin, cajanin, orobol, pratensein, santal, junipegenin A, glycitein, afrormosin, retusin, tectorigenin, irisolidone, jamaicin, and their analogs and/or metabolites.

According to the present invention, among the isoflavonoids, it is preferable to use the isoflavones. Most particularly, it is preferable to use, according to the invention, the simplest aglycone forms among which daidzein, genistein and mixtures thereof. These two compounds are in particular present in the soybean (Glycina max) extract available from Archer Daniels Midland Company under the name Novasoy®.

The quantity of isoflavonoid which can be used according to the invention of course depends on the desired effect and may therefore vary to a large degree.

To give an order of magnitude, in the composition according to the invention, the isoflavonoid in the pure state is in a quantity representing from 10⁻¹⁰% to 10% of the total weight of the composition, and preferably in a quantity representing from 10⁻⁸% to 5% of the total weight of the composition.

Of course, if the isoflavonoid is present in the form of a solution, for example a plant extract, persons skilled in the art will know how to adjust the quantity of this solution in the composition according to the invention, so as to obtain the isoflavonoid concentration ranges described above.

Methods for preparing isoflavones are in particular described in WO 95/10530, WO 95/10512, U.S. Pat. Nos. 5,679,806, 5,554,519, EP-812,837 and WO 97/26269. Isoflavones are in particular known as antioxidants, for their anti-free radical and depigmenting properties, and for inhibiting the activity of the sebaceous glands (see in particular DE-44,32,947).

The composition of the invention may be in all the galenic forms imaginable, suitable both for topical application to the skin and/or the mucous membranes and/or head hair and/or body hair and for administration by the oral route. Preferably, the composition of the invention is intended for administration by the oral route.

The composition of the invention may be a cosmetic or dermatological composition. Preferably, according to the invention, the composition is a cosmetic composition. The composition is a cosmetic composition because it is intended to improve the general skin appearance of the individual using it. Most preferably, the composition of the invention is a cosmetic composition intended for administration by the oral route.

For administration by the oral route, the composition of the invention may be provided in all the suitable forms, particularly in the form of an oral solution, a syrup, a tablet, a sugar-coated tablet, a hard gelatin capsule or a soft gelatin capsule or a nutritional food or a nutritional supplement. Said composition may comprise, in addition, at least one appropriate excipient suitable for oral administration.

For administration by topical application to the skin, head hair and/or body hair and/or the mucous membranes, the composition according to the invention quite obviously comprises a cosmetically acceptable carrier, that is to say a carrier compatible with the skin, the mucous membranes, the nails, head hair and body hair, and may be provided in all the galenic forms normally used for a topical application, in particular in the form of an aqueous, aqueous-alcoholic or oily solution, an oil-in-water or water-in-oil or multiple emulsion, an aqueous or oily gel, an anhydrous liquid, pasty or solid product, a dispersion of oil in an aqueous phase with the aid of spherules, it being possible for these spherules to be polymeric nanoparticles such as nanospheres and nanocapsules or better still lipid vesicles of the ionic and/or nonionic type.

This composition may be fluid to a greater or lesser degree and may have the appearance of a white or colored cream, an ointment, a milk, a lotion, a serum, a paste, a foam. It may be optionally applied to the skin in the form of an aerosol. It may also be provided in solid form, and for example in the form of a stick. It may be used as a treatment product, as a cleansing product, as a make-up product or alternatively as a simple deodorant product.

In a known manner, the composition of the invention may also contain the customary adjuvants in the cosmetic and dermatological fields, such as hydrophilic or lipophilic gelling agents, hydrophilic or lipophilic active agents, preservatives, antioxidants, solvents, perfumes, fillers, screening agents, pigments, chelating agents, odor absorbers and coloring matter. The quantities of these various adjuvants are those conventionally used in the fields considered, and for example from 0.01 % to 20% of the total weight of the composition. These adjuvants, depending on their nature, may be introduced into the fatty phase, into the aqueous phase, into the lipid vesicles and/or into the nanoparticles.

When the composition of the invention is an emulsion, the proportion of the fatty phase may range from 5% to 80% by weight, and preferably from 5% to 50% of the total weight of the composition. The oils, the emulsifiers and the coemulsifiers used in the composition in the form of an emulsion are chosen from those conventionally used in the field considered. The emulsifier and the coemulsifier are present in the composition in a proportion ranging from 0.3% to 30% by weight, and preferably from 0.5% to 20% of the total weight of the composition.

As oils which can be used in the invention, there may be mentioned mineral oils, oils of plant origin (apricot oil, sunflower oil), oils of animal origin, synthetic oils, silicone oils and fluorinated oils (perfluoropolyethers). It is also possible to use, as fats, fatty alcohols (cetyl alcohol), fatty acids, waxes (beeswax).

As emulsifiers and coemulsifiers which can be used in the invention, there may be mentioned for example esters of fatty acid and polyethylene glycol such as PEG-40 stearate, PEG-100 stearate, esters of fatty acid and polyol such as glyceryl stearate and sorbitan tristearate.

As hydrophilic gelling agents, there may be mentioned in particular carboxyvinyl polymers (carbomer), acrylic copolymers such as copolymers of acrylates/alkyl acrylates, polyacrylamides, polysaccarides, natural gums and clays, and, as lipophilic gelling agents, there may be mentioned modified clays such as bentones, metal salts of fatty acids, hydrophobic silica and polyethylenes.

The composition may contain other hydrophilic active agents such as proteins or protein hydrolysates, amino acids, polyols, urea, allantoin, sugars and sugar derivatives, plant extracts and hydroxy acids.

As lipophilic active agents, there may be used retinol (vitamin A) and its derivatives, tocopherol (vitamin E) and its derivatives, essential fatty acids, ceramides, essential oils, salicylic acid and its derivatives or vitamins B1, B6 and/or B12.

It is also possible to use in the composition of the invention vitamin C (or ascorbic acid) and its derivatives (esters, salts, and the like).

A preferred composition of the invention comprises, among other ingredients, the combination of at least one carotenoid and at least one isoflavonoid, an extract of vitamin C and of α-tocopherol.

Still more preferably, the composition of the invention comprises, among other ingredients, the combination of β-carotene, of soybean extract (Novasoy® from Archer Daniels Midland Company), of vitamin C and of atocopherol acetate.

It is also possible to use, in addition, in the composition of the invention, compounds chosen from

trace elements;

plant hormones;

calcium antagonists, such as verapamil and diltiazem;

scavengers of OH radicals, such as dimethyl sulfoxide;

chlorine channel openers;

plant extracts, microbial extracts including in particular bacterial extracts such as those of nonphotosynthetic filamentous bacteria.

Other compounds may also be added to the above list, namely for example potassium channel openers such as diazoxide and minoxidil, spiroxazone, phospholipids such as lecithin, linoleic and linolenic acids, salicylic acid and its derivatives disclosed in FR 2,581,542, such as the salicylic acid derivatives carrying an alkanoyl group having from 2 to 12 carbon atoms at the 5-position of the benzene ring, hydroxycarboxylic or ketocarboxylic acids and their esters, lactones and their corresponding salts, anthralin, carotenoids, eicosatetraenoic and eicosatrienoic acids or their esters and amides, vitamin D and its derivatives.

According to the invention, other active agents intended especially for the prevention and/or treatment of skin conditions may be added, inter alia, to the composition of the invention. Among these active agents, there may be mentioned, by way of example:

agents modulating skin differentiation and/or proliferation and/or pigmentation such as retinoic acid and its isomers, retinol and its esters, vitamin D and its derivatives, oestrogens such as oestradiol, kojic acid or hydroquinone;

antibacterials such as clindamycin phosphate, erythromycin or antibiotics of the tetracycline class;

agents modulating bacterial adhesion to the skin and/or mucous membranes such as honey, especially honey from acacias and certain sugar derivatives;

antiparasitic agents, in particular metronidazole, crotamiton and pyrethrinoids;

antifungal agents, in particular the compounds belonging to the imidazole class such as econazole, ketoconazole or miconazole or their salts, polyene compounds, such as amphotericin B, compounds of the allylamine family, such as terbinafine, or octopirox;

antiviral agents such as acyclovir;

steroidal anti-inflammatory agents such as hydro-cortisone, betamethasone valerate or clobetasol propionate, or nonsteroidal anti-inflammatory agents such as ibuprofen and its salts, diclofenac and its salts, acetylsalicylic acid, acetaminophen or glycyrrhetinic acid;

anaesthetic agents such as lidocain hydrochloride and its derivatives;

antipruriginous agents such as thenaldine, trimeprazine or cyproheptadine;

keratolytic agents such as alpha- and beta-hydroxycarboxylic or betaketocarboxylic acids, their salts, amides or esters and more particularly hydroxy acids such as glycolic acid, lactic acid, salicylic acid, citric acid and, in general, fruit acids and 5-n-octanoylsalicylic acid;

anti-free radical agents, such as superoxide dismutases, certain metal chelators or ascorbic acid and its esters;

antiseborrheic agents such as progesterone;

antidandruff agents such as octopirox or zinc pyrithione;

anti-acne agents such as retinoic acid or benzoyl peroxide;

substances such as substance P, CGRP or bradykinin antagonists or NO synthase inhibitors or alternatively sodium channel inhibitors, compounds described as being active in the treatment of sensitive skins and as having anti-irritant effects, in particular toward irritant compounds which may be present in the compositions.

As active agents, it is possible to use in particular moisturizers such as polyols (for example glycerine), vitamins (for example D-panthenol), anti-inflammatory agents, soothing agents (allantoin, cornflower water), UVA- and UVB-screening agents, matting agents (for example partially cross-linked polydimethylorganosiloxanes sold under the name KSG® by Shin Etsu), and mixtures thereof.

It is also possible to add antiwrinkle active agents and in particular toning products such as vegetable proteins and their hydrolysates, in particular the extract of soybean proteins sold under the name Eleseryl® by LSN or the oats derivative sold under the name Reductine® by Silab.

As the skin consists of many other components apart from collagen and the fibroblasts, it is advantageous, when the combination of the invention is used, to promote at the same time the synthesis of these other components such as for example lipids and/or to promote the proliferation of other cellular components such as for example the keratinocytes.

Thus, the subject of the invention is a cosmetic composition comprising, in a cosmetically acceptable medium, the combination of at least one carotenoid and at least one isoflavonoid and at least one other product stimulating the synthesis of lipids and/or the proliferation of keratinocytes.

In this composition, the carotenoid and the isoflavonoid may be as described as above in the text.

Said composition may be intended for use in the cosmetic or dermatological field, preferably in the cosmetic field.

There may be mentioned, as product stimulating the synthesis of lipids, plant hormones, such as auxins, or compounds of plant origin, such as cinnamic acid, and as product stimulating proliferation of keratinocytes, compounds of plant origin, such as phloroglucinol.

Thus the compositions according to the invention may also comprise the combination of the invention, cinnamic acid or its derivatives and/or a plant hormone, in particular an auxin chosen from indoleacetic acid (IAA), 4-chloroindole-3-acetic acid (4-CI-IAA), phenylacetic acid (PAA), indole-3-butyric acid (IBA), 2,4-dichlorophenoxyacetic acid (2,4-D), α-naphthaleneacetic acid (α-NAA), β-naphthoxyacetic acid, indoleethanol, idoleacetaldehyde and indoleacetonitrile and/or a plant compound such as phloroglucinol.

The subject of the invention is also the use of the combination of at least one carotenoid, of at least one isoflavonoid and of at least one other product stimulating the synthesis of lipids and/or the proliferation of keratinocytes in a composition or for the preparation of a composition, the combination or the composition being intended for treating, preventively and/or curatively, the cutaneous signs of aging, for combating the degradation of collagen, for inhibiting the expression of the proteases of the extracellular matrix, for inhibiting the expression of metalloproteinases, particularly metalloproteinase type 1, for treating skin conditions linked to the menopause, for combating wrinkles and fine lines, for combating withered skin, for combating soft skin, for combating skin with reduced thickness, for combating dull skin and/or skin with no brightness, for combating the lack of elasticity and/or of tone of the skin, for combating internal damage to the skin following exposure to ultraviolet radiation.

In this regard, there may be mentioned, as product stimulating the synthesis of lipids, plant hormones, such as auxins, or compounds of plant origin, such as cinnamic acid, and, as product stimulating proliferation of keratinocytes, compounds of plant origin, such as phloroglucinol.

Thus the compositions according to the invention may also comprise the combination of the invention, cinnamic acid or its derivatives and/or a plant hormone, in particular an auxin chosen from indoleacetic acid (IAA), 4chloroindole-3-acetic acid (4-CI-IAA), phenylacetic acid (PAA), indole-3-butyric acid (IBA), 2,4-dichlorophenoxyacetic acid (2,4-D), α-naphthaleneacetic acid (α-NAA), β-naphthoxyacetic acid, indoleethanol, idoleacetaldehyde and indoleacetonitrile and/or a plant compound such as phloroglucinol.

The subject of the present invention is a method for the cosmetic treatment of the skin intended for stimulating the synthesis of collagen and/or for combating skin conditions linked to age and/or to the menopause and/or for combating the reduction in the thickness of the dermis and/or for combating the appearance of soft and/or wrinkled skin, characterized in that there is applied to the skin, to head hair, to body hair and/or to the mucous membranes or there is ingested a cosmetic composition comprising at least one combination of at least one carotenoid and of at least one isoflavonoid.

The method of cosmetic treatment of the invention may be carried out in particular by applying the cosmetic compositions as defined above, according to the usual technique for using these compositions. For example: application of creams, gels, sera, lotions, milks, shampoos or compositions for protecting against sunlight, to the skin or to head hair or to body hair or alternatively application of dentifrice to the gums and preferably by administration, by the oral route, of an oral solution, a syrup, a tablet, a hard gelatin capsule, a soft gelatin capsule or a nutritional food or a nutritional supplement.

In order to further illustrate the present invention and the advantages thereof, the following specific examples are given, it being understood that same are intended only as illustrative and in nowise limitative.

In said examples to follow, all parts and percentages are given by weight.

### EXAMPLES

### Composition 1—Soft Gelatin Capsules

| | |
|---|---|
| Excipients: | |
| Soybean oil | 40 mg |
| Wheatgerm oil | 85 mg |
| Soybean lecithins | 25 mg |
| Vitamin: | |
| Natural tocopherols | 3 mg |
| Vitamin C | 60 mg |
| Components: | |
| Lycopene | 6 mg |
| Novasoy ® - soybean extract containing | 120 mg |
| isoflavones (50 mg of isoflavones per 120 mg of Novasoy ®) | |

### Composition 2: Lotion

| | | |
|---|---|---|
| Lycopene at 6% (Lycomato ®) | | 0.001% |
| Isoflavones (Novasoy ®) | | 0.10% |
| Antioxidant | | 0.05% |
| Isopropanol | | 40.0% |
| Preservative | | 0.30% |
| Water | qs | 100.00 |

### Composition 3: Care Cream (Oil-in-Water Emulsion)

| | | |
|---|---|---|
| Lycopene at 6% (Lycomato ®) | | 0.005% |
| Soybean extract (Novasoy ®) | | 0.10% |
| Vitamin C | | 5.00% |
| Glyceryl stearate | | 2.00% |
| Polysorbate 60 (Tween 60 ® sold by ICI) | | 1.00% |
| Stearic acid | | 1.40% |
| Triethanolamine | | 0.70% |
| Carbomer | | 0.40% |
| Liquid fraction of shea butter | | 12.00% |
| Perhydrosqualene | | 12.00% |
| Antioxidant | | 0.05% |
| Perfume | | 0.50% |
| Preservative | | 0.30% |
| Water | qs | 100.00% |

### Composition 4: Care Cream (Oil-in-Water Emulsion)

| | | |
|---|---|---|
| Lycopene at 6% (Lycomato ®) | | 0.001% |
| Soybean extract (Novasoy ®) | | 0.10% |
| Glyceryl stearate | | 2.00% |
| Polysorbate 60 (Tween 60 ® sold by ICI) | | 1.00% |
| Stearic acid | | 1.40% |
| Glycyrrhetinic acid | | 2.00% |
| Triethanolamine | | 0.70% |
| Carbomer | | 0.40% |
| Liquid fraction of shea butter | | 12.00% |
| Sunflower oil | | 10.00% |
| Antioxidant | | 0.05% |
| Perfume | | 0.50% |
| Preservative | | 0.30% |
| Water | qs | 100.00% |

Each patent, patent application and -literature article/report cited or indicated herein is hereby expressly incorporated by reference.

While the invention has been described in terms of various specific and preferred embodiments, the skilled artisan will appreciate that various modifications, substitutions, omissions, and changes may be made without departing from the spirit thereof. Accordingly, it is intended that the scope of the present invention be limited solely by the scope of the following claims, including equivalents thereof.

What is claimed is:

1. A regime or regimen for treating the cutaneous signs of skin aging, comprising administering to a mammal in need of such treatment, a thus effective amount of a cosmetic/dermatological composition which comprises intimate admixture of at least one carotenoid and at least one isoflavonoid.

2. A regime or regimen for combating the degradation of collagen, comprising administering to a mammal in need of such treatment, a thus effective amount of a cosmetic/dermatological composition which comprises intimate admixture of at least one carotenoid and at least one isoflavonoid.

3. A regime or regimen for inhibiting the expression of the proteases of the extracellular matrix, comprising administering to a mammal in need of such treatment, a thus effective amount of a cosmetic/dermatological composition which comprises intimate admixture of at least one carotenoid and at least one isoflavonoid.

4. The regime or regimen as defined by claim 3, for inhibiting the expression of metalloproteinases.

5. The regime or regimen as defined by claim 4, for inhibiting the expression of metalloproteinase type 1.

6. A regime or regimen for treating conditions of the skin resulting from menopause, comprising administering to a mammal in need of such treatment, a thus effective amount of a cosmetic/dermatological composition which comprises intimate admixture of at least one carotenoid and at least one isoflavonoid.

7. A regime or regimen for combating skin wrinkles and fine lines, comprising administering to a mammal in need of such treatment, a thus effective amount of a cosmetic/dermatological composition which comprises intimate admixture of at least one carotenoid and at least one isoflavonoid.

8. A regime or regimen for combating withered skin, comprising administering to a mammal in need of such treatment, a thus effective amount of a cosmetic/dermatological composition which comprises intimate admixture of at least one carotenoid and at least one isoflavonoid.

9. A regime or regimen for combating soft skin, comprising administering to a mammal in need of such treatment, a thus effective amount of a cosmetic/dermatological composition which comprises intimate admixture of at least one carotenoid and at least one isoflavonoid.

10. A regime or regimen for combating skin with a reduced thickness, comprising administering to a mammal in need of such treatment, a thus effective amount of a cosmetic/dermatological composition which comprises intimate admixture of at least one carotenoid and at least one isoflavonoid.

11. A regime or regimen for combating dull skin or skin with no brightness, comprising administering to a mammal in need of such treatment, a thus effective amount of a cosmetic/dermatological composition which comprises intimate admixture of at least one carotenoid and at least one isoflavonoid.

12. A regime or regimen for treating skin damaged by exposure to ultraviolet radiation, comprising administering to a mammal in need of such treatment, a thus effective amount of a cosmetic/dermatological composition which comprises intimate admixture of at least one carotenoid and at least one isoflavonoid.

13. A regime or regimen for inhibiting the activity and/or the expression of collagenases and by increasing the synthesis of collagen, comprising administering to a mammal in need of such treatment, a thus effective amount of a cosmetic/dermatological composition which comprises intimate admixture of at least one carotenoid and at least one isoflavonoid.

14. A cosmetic/dermatological composition suited for treating the undesirable cutaneous signs of skin aging, comprising intimate admixture of thus effective amounts of at least one carotenoid and at least one isoflavonoid, formulated into a cosmetically/dermatologically acceptable carrier therefor.

15. The cosmetic/dermatological composition as defined by claim 14, said at least one carotenoid comprising a carotenoid having provitamin A activity, a carotenoid devoid of provitamin A activity, or mixture thereof.

16. The cosmetic/dermatological composition as defined by claim 15, said at least one carotenoid comprising β-carotene, α-carotene, or mixture thereof.

17. The cosmetic/dermatological composition as defined by claim 16, said at least one carotenoid comprising β-carotene.

18. The cosmetic/dermatological composition as defined by claim 15, said at least one carotenoid comprising zeaxanthin, cryptoxanthin, lutein, lycopene, or mixture thereof.

19. The cosmetic/dermatological composition as defined by claim 18, said at least one carotenoid comprising lycopene.

20. The cosmetic/dermatological composition as defined by claim 14, said at least one carotenoid comprising from 10⁻¹²% to 20% by weight thereof.

21. The cosmetic/dermatological composition as defined by claim 14, said at least one carotenoid comprising from 10⁻¹⁰% to 10% by weight thereof.

22. The cosmetic/dermatological composition as defined by claim 14, said at least one isoflavonoid comprising an isoflavone, isoflavanone, retinoid, pterocarpan, isoflavan, isoflavan-3-ene, 3-arylcoumarin, 3-aryl-4-hydroxycoumarin, coumestan, coumaronochromone, α-methyldeoxybenzoin, 2-arylbenzofuran, or mixture thereof.

23. The cosmetic/dermatological composition as defined by claim 14, said at least one isoflavonoid comprising daidzein, formononetin, cuneatin, genistein, isoprunetin and prunetin, cajanin, orobol, pratensein, santal, junipegenin A, glycitein, afrormosin, retusin, tectorigenin, irisolidone, jamaicin, analog and/or metabolite or mixture thereof.

24. The cosmetic/dermatological composition as defined by claim 14, said at least one isoflavonoid comprising an isoflavone.

25. The cosmetic/dermatological composition as defined by claim 14, said at least one isoflavonoid comprising diadzein, genistein, or mixture thereof.

26. The cosmetic/dermatological composition as defined by claim 14, said at least one isoflavonoid comprising from 10⁻¹⁰% to 10% by weight thereof.

27. The cosmetic/dermatological composition as defined by claim 26, said at least one isoflavonoid comprising from 10⁻⁸% to 5% by weight thereof.

28. A cosmetic/dermatological composition suited for treating the undesirable cutaneous signs of skin aging, comprising intimate admixture of thus effective amounts of at least one carotenoid, at least one isoflavonoid and at least one other active agent for stimulating the synthesis of lipids and/or the proliferation of the keratinocrytes, formulated into a cosmetically/dermatologically acceptable carrier therefor.

29. The cosmetic/dermatological composition as defined by claim 28, comprising at least one other active agent for stimulating the synthesis of lipids and which comprises a plant hormone, an auxin, a compound of plant origin, cinnamic acid or derivative thereof, or mixture thereof.

30. The cosmetic/dermatological composition as defined by claim 29, comprising indoleacetic acid (IAA), 4-chloroindole-3-acetic acid (4-CI-IAA), phenylacetic acid (PAA), indole-3-butyric acid (IBA), 2,4-dichlorophenoxyacetic acid (2,4-D), α-naphthaleneacetic acid (α-NAA), β-naphthoxyacetic acid, indoleethanol, idoleacetaldehyde, indoleacetonitrile, or mixture thereof.

31. The cosmetic/dermatological composition as defined by claim 28, comprising phloroglucinol.

32. An orally administratable cosmetic/dermatological composition suited for treating the undesirable cutaneous signs of skin aging, comprising intimate admixture of thus effective amounts of at least one carotenoid and at least one isoflavonoid, formulated into an orally administratable, cosmetically/dermatologically acceptable carrier therefor.

33. The cosmetic/dermatological composition as defined by claim 32, formulated as a solution, syrup, tablet, gelatin capsule, or nutritional food or supplement.

34. A topically applicable cosmetic/dermatological composition suited for treating the undesirable cutaneous signs of skin aging, comprising intimate admixture of thus effective amounts of at least one carotenoid and at least one isoflavonoid, formulated into a topically applicable, cosmetically/dermatologically acceptable carrier therefor.

35. The cosmetic/dermatological composition as defined by claim 34, formulated as a solution, emulsion, gel, paste, solid, spherules, vesicles, cream, ointment, milk, lotion, serum, foam, aerosol, makeup, or deodorant.
